(19) United States
(12) Patent Application Publication
(10) Pub. No.: US 2010/0021340 A1
(43) Pub. Date: Jan. 28, 2010

(54) Method and device for the disinfection of objects

(75) Inventors: Christian Buske, Steinhagen (DE); Peter Förnsel, Spenge (DE); Joachim Wunderlich, Buch Am Erlbach (DE)

Correspondence Address: PROSKAUER ROSE LLP, ONE INTERNATIONAL PLACE, BOSTON, MA, 02110, US

(73) Assignee: PlasmaTreat GmbH, Steinhagen (DE)

(21) Appl. No.: 12/097,987

(22) PCT Filed: Dec. 20, 2006
(86) PCT No.: PCT/EP2006/069997

§ 371 Date: Nov. 24, 2008

(30) Foreign Application Priority Data
Dec. 20, 2005 (DE) 10 2005 061 236.9

(51) Int. Cl. A61L 2/20

(52) U.S. Cl. 422/28

(57) ABSTRACT

A method for the disinfection of objects includes producing an atmospheric plasma jet inside a space through which a process gas flows and at least partially bombarding a surface of the object with the plasma jet, whereby a disinfecting effect is achieved by way of an energy transfer from the plasma jet to the surface of the object. With this method a more effective surface disinfection is made possible without increasing the quantity of disinfecting agent in itself.

### CROSS-REFERENCE TO RELATED APPLICATIONS

This application is a National Phase Application of International Application No. PCT/EP2006/069997, filed on Dec. 20, 2006, which claims the benefit of and priority to German patent application no. DE 10 2005 061 236.9, filed Dec. 20, 2006. The disclosure of the above applications are incorporated herein by reference in their entirety.

### FIELD OF THE INVENTION

The invention is related to a procedure for the disinfection of objects.

### BACKGROUND

In numerous industrial and laboratory-technical procedures good disinfection of the used objects is important.

Disinfection is the reduction of the number of viable microorganisms with the aid of physical or chemical methods. It is the aim of disinfection to make certain pathogenic microorganisms harmless by intervention in their structure or metabolism.

The applications of disinfection are mainly concerned with surface disinfection and room disinfection, device disinfection and instrument disinfection as well as the disinfection of clothing. These applications are necessary above all in the medical field, but may also be necessary in other non-medical applications.

Disinfecting agents known in prior art are often very aggressive to surfaces, so that they are only allowed to be handled using protective measures, such as protective gloves.

In addition, the breathing of the gases evaporating from concentrated liquids may also cause health hazards.

The previously mentioned disinfecting substances are often applied to the surface in great concentrations. It is usually left to the user as to how much of the disinfecting agent he applies. High concentrations are often necessary, because the disinfecting agent cannot become sufficiently effective.

Further disinfecting methods are based on the application of radiation, such as UV-radiation or even radioactive radiation. However, these methods have either the disadvantage of low effectivity or require a high technical effort.

### SUMMARY OF THE INVENTION

The technical problem of the invention is therefore to find a procedure for the disinfection by eliminating the previously indicated disadvantages.

In a procedure for the disinfection of surfaces in accordance with an embodiment of the invention, an atmospheric plasma jet is produced by the use of an electrical discharge in space through which a process gas flows and the surface is at least partially exposed to the plasma jet. The disinfecting effect occurs then via the energy transfer from the plasma jet to the surface.

Embodiments of the invention are also based on the knowledge that by using an atmospheric plasma jet a reactive agent is brought into contact with the surface of the object, which on one hand has a high reactivity due to a highly excited electrons and on the other side has a low gas temperature. The high reactivity is utilized for the disinfection by at least partially, but preferably mostly destroying microorganisms present on the surfaces due to the electron reactivity.

At the same time the surface is exposed to only slight thermal stress by the plasma jet, so that the surface itself is not damaged or changed. Therefore even delicate surfaces can be thoroughly disinfected, which to date were not able to be exposed to the above mentioned aggressive disinfecting agents.

Another benefit of the procedure in accordance with the invention is in the fact that the plasma jet—in opposition to the known applications of liquid disinfection agents—is able to disinfect not only smooth surfaces, but also those three dimensional structures that are difficult to disinfect without residues remaining in comers and creases. Due to the gas flow, the plasma jet prevents the remaining of impurities in these areas.

The previously described procedures as well as the devices described below, utilize preferably the plasma sources or plasma nozzles described below, which represent the above mentioned space through which an process gas flows.

The energy supply for the plasma disinfection is preferably produced using a plasma source or a plasma nozzle, in which a plasma jet is produced by a non-thermal discharge from a process gas by application of a high-frequency high voltage in a nozzle pipe between two electrodes. During this process the process gas is preferably under pressure in the magnitude of atmospheric pressure. Therefore the term atmospheric plasma is used.

The plasma jet exits a nozzle opening, whereby one of the two electrodes is arranged within the area of the nozzle opening. When a suitable flow rate is achieved, the non-thermic plasma jet has no electrical streamers outside the plasma nozzle, or discharge channels of the electrical discharge, so that only the energy rich, low temperature plasma jets is directed at the surface. Such an atmospheric plasma jet is also referred to as potential-free plasma jet. The potential difference between the nozzle opening and the work piece is preferably below 100 v.

In order to characterize the gas properties of the plasma jet, high electron temperatures and low ion temperatures are mentioned. High electron temperatures cause high reactivity of the plasma gas or the plasma gas mixture. The low ion temperature on the other hand causes a low heat energy, which is transferred to the surface during the contact with the plasma jet.

Such plasma sources are known in prior art in EP 0 761 415 A1 and EP 1 335 641 A1. For applications on larger surfaces, however, the rotation nozzles known from WO 99/52333 and WO 01/43512 are more suitable.

The plasma jet is preferably produced with the aid of an atmospheric discharge in an process gas containing oxygen. This increases the reactivity of the plasma. Air is used in preference as process gas. Likewise, the process gas may also consist of a mixture of hydrogen and nitrogen, also referred to as inert gas. Pure nitrogen may also be used as process gas.

Non-thermic plasma discharge occurs particularly by the application of high frequency high voltage, whereby a series of discharges is produced between two electrodes of the plasma nozzle and the process gas is excited into a plasma exiting from the plasma nozzle. In particular the high frequency of discharges guarantees that a thermic equilibrium is prevented within the discharge space. Therefore the disequilibrium between electron temperature and ion temperature is maintained.

The effectivity of the plasma treatment naturally also depends on the choice of the process gas, the output, the treatment duration and the construction concept and adjustments may be made, if necessary. In particular the potential parameters, frequency and amplitude represent suitable means to affect the effectivity of the plasma treatment.

In the prior art of DE 37 33 492, the production of the atmospheric plasma jet occurs with the aid of a corona discharge due to an ionization of the process gas, for example air. The device consists of a ceramic tube, which is surrounded on the outer wall with an outer electrode. An inner rod-shaped electrode is arranged only a few millimeters away from the inner wall of the ceramic tube. An ionizable gas, such as air or oxygen, is conducted through the gap between the inner wall of the ceramic tube and the inner electrode. A high frequency high voltage is applied to both electrodes, as used in the corona pretreatment of foils. Due to the alternating field, the conducted gas is ionized and exits at the end of the tube.

Finally, it does not depend on how the process gas was excited in order to produce the plasma, as long as an atmospheric plasma jet with sufficient intensity can be produced.

The previously described procedures enable the above mentioned application in accordance with the invention for the disinfection of objects.

Preferably, the sterilizing effect of the plasma jet can be enhanced by adding disinfecting agents to the volume flooded by the process gas. This disinfecting agent is excited due to the discharge and/or a plasma jet and therefore can have an enhanced disinfecting effect on the surface of the object.

The excitation of the disinfecting agent refers to excitation of electrons and resulting ionization or dissociation. These excited conditions in particular enable a extremely effective energy transfer from the plasma jet to the surface of the object and therefore increase the disinfecting effect of the plasma jet itself.

Gaseous substances are added in preference as disinfecting agents to the process gas or the plasma jet. Likewise, liquid disinfecting agents may be added to the mixture. If liquid disinfecting agents are added, it is preferred that these are sprayed, misted or injected into the space through which the process gas flows in order to distribute the liquid as even as possible within the process gas or the plasma jet.

The disinfecting agent may be chosen from the group described below:
Aldehydes, such as formaldehyde, glutaraldehyde, glycoxal.
Alcohols, such as ethanol, propanol, isopropanol.
Per-compounds, such as hydrogen peroxide, per-acetic acid, potassium peroxomonosulfate.
Halogens, such as sodium hypochlorite, chlorine dioxide, sodium chlorite, chloramine.
Halogenised phenols, such as m-kresol, p-chloro-mkresol, p-chloro-m-xylenol.
Quaternary ammonium compounds, such as benzalkonium.

Water may also be added as liquid disinfecting agent, because a mixture of process gas water vapor is produced due to the excitation in and/or due to the plasma. This mixture also has a disinfecting effect, even if water in itself at normal temperatures does not exhibit a disinfecting effect.

In general, the disinfecting agent may be introduced into the space in three different areas.

In a first embodiment, the disinfecting agent may be mixed into the process gas upstream of the discharge zone. This may be achieved for example in two ways. Either, the disinfecting agent may have already been added to the process gas, or the disinfecting agent may be introduced in the vicinity of the inlet of the process gas with a separate inlet.

In another embodiment of the invention, the disinfecting agent is added to the process gas in the area of the discharge zone. Here also two possibilities are available. Either the disinfecting agent may be introduced into the space to the side of the discharge zone by a separate inlet, or the disinfecting agent may be introduced through an opening, which is situated within one of the electrodes provided for the electrical discharge. This enables the accurate placement of the disinfecting agent into the discharge zone.

In yet another embodiment, the disinfecting agent may be mixed into the process gas downstream of the discharge zone. This procedure is preferable in situations where the disinfecting agent is intended to be exposed not to the discharge, but rather the plasma jet itself, which has a far greater effect. In order to achieve this, the disinfecting agent may be added with the aid of an inlet or lance arranged inside the plasma jet.

In the previously discussed variations of the procedure plasma nozzles may be used, which are already known in prior art for the plasma surface coating or plasma polymerization. These nozzle types have in common, that a precursor necessary for the plasma polymerization is injected into the space of the electrical discharge at various points. This is referred to for example in EP 1 230 414 and DE 100 61 828.

### BRIEF DESCRIPTION OF THE DRAWINGS

In the following, an embodiment of the present invention, namely an applicable plasma nozzle, which is suitable for the execution of the invented procedure, is described and explained and which is illustrated by FIG. 1 (of 1):

There is shown in:

FIG. 1 an embodiment of a plasma nozzle, which can be applied in the invented procedure.

### DESCRIPTION

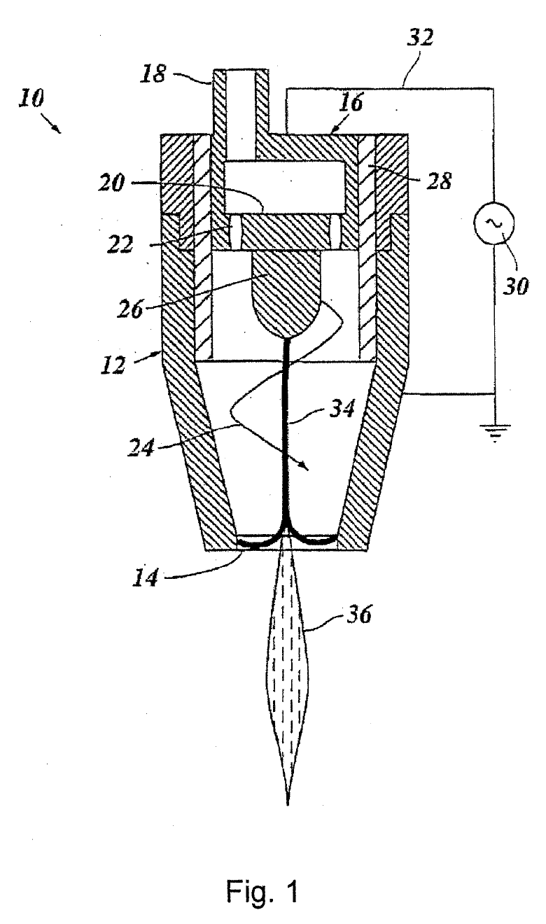

A source of plasma, as shown in FIG. 1, is plasma nozzle 10, possessing a metallic outer housing 12 of, in this case, circular cross-section, part of which tapers conically to terminate in a discharge opening 14. On that end of the nozzle housing 12, which is remote from the discharge opening 14, is located a swirl chamber 16, which possesses an inlet 18 for process gas. For example this process gas can be compressed air or nitrogen.

The plasma nozzle 10 of FIG. 1 is furnished with a centrally aligned discharge opening 14. Further, in this respect, an alternate possibility exists, that the said discharge opening 14 can be inclined angularly to its central axis and additionally be capable of rotating in transversely to the axis of the said nozzle housing 12. In a case wherein a rotational movement of the said discharge opening 14 is achieved, one advantage is that the process gas is subjected to additional swirling. Such a rotational nozzle has been made known by EP 1 067 829 A2.

A transversely, horizontally situated partition 20, which is an integral part of the swirl chamber 16, possesses a circumferentially disposed ring of inclined borings 22. These borings impart to the process gas a primary swirling motion. The conically tapered, downstream part of the nozzle 10 is consequently subjected to a flow of spirally swirling process gas, denoted by reference number 24. The central axis of the said spiral gas swirl 24 is also the vertical axis of the nozzle 10.

Centrally placed on the downstream side of the partition 20, is to be found an electrode 26. This electrode 26 extends itself coaxially into the internal space of the plasma nozzle housing 12. Electrical communication binds the electrode 26 with the partition 20 and the thereto connected parts of the swirl chamber 16. However, the swirl chamber 16 is insulated from the nozzle housing 12 by means of a concentric, ceramic tube 28, which forms a liner within the cylindrical section of the nozzle housing 12. The described electrode 26, being located downstream of the partition 20, is designed to accommodate high frequency, high voltage, alternating electrical current from a frequency converter 30. Direct current may also be employed with suitable circuitry.

The primary voltage can be variably controlled and normally falls within a range of 300 to 500 volts. The secondary voltage runs between 1 to 5 kV (or higher), the voltage being measured peak to peak. For example, the frequency lies between a general magnitude of 1 to 100 kHz and again is controllable through a variable range. The applied frequency may also lie outside of the values given here, as long as an arc-initiation discharge value is maintained. The swirl chamber 16 is connected to the said high frequency converter 30 by a flexible, high voltage cable 32. The inlet port 18 is connected by means of a (not shown) tube to a pressurized process gas source, which source produces a variably controlled flow. Advantageously, in order to provide a reliable source of supply, the control of the process gas source and that of the high frequency converter 30 are electrically combined.

By means of the stated applied voltage, a high frequency discharge in the form of an arc 34 is established between the electrode 26 and the grounded nozzle housing 12. The word “arc” is employed as a phenomenological adjective describing said arc path, which is in the form of a visible discharge. This arc, in the case of the already mentioned possibility of direct current discharge, is to be understood as being formed at an essentially constant voltage. Due to the imparted swirling of the process gas, the arc follows a helical path about the longitudinal axis of the nozzle housing 12. The result of this is, that the arc cannot branch off into the wall of the nozzle housing 12 until it is immediately proximal to the rim of the discharge opening 14. Entrained in this spiral flow zone, the process gas is accelerated in the neighborhood of the arc 34 and being thus in intimate contact therewith, is converted into a plasma condition. In this way, a jet 36 of atmospheric plasma is then caused to be emitted from the discharge opening 14 of the nozzle 10, carrying the appearance of a candle flame.

The plasma nozzle, which has been described and explained above, can be applied to the enveloping of the object to be disinfected. In regard to the object to be disinfected, this may be, for example, part or whole of medicinal treatment equipment, a working surface or the exposed surface of a food handling container. During a period in which the plasma jet 36 emerges from the above said nozzle discharge opening 14, the imparted alternating treatment of the plasma gas with the to-be-disinfected surface is maintained, whereby the action of the disinfecting, sterilizing and germicidal action of the nozzle 12 is achieved.

1. Method for disinfection of objects
utilizing an electric discharge in a space through which a process gas flows to produce an atmospheric plasma jet, and
exposing a surface of an object at least partially to the atmospheric plasma jet,
wherein a disinfecting effect occurs due to an energy transfer from the atmospheric plasma jet to the surface.

2. Method according to claim 1, wherein a disinfecting agent is added into the space through which the process gas flows.

3. Method according to claim 2, wherein a gas is added as disinfecting agent.

4. Method according to claim 2, wherein a liquid is added as disinfecting agent.

5. Method according to claim 4, during which the liquid disinfecting agent is sprayed or misted into the space through which the process gas flows or injected with the aid of nozzles.

6. Method according to claim 2, wherein the disinfecting agent is mixed into the process gas upstream of the a discharge zone.

7. Method according to claim 2, wherein the disinfecting agent is mixed into the process gas in an area of a discharge zone.

8. Method according to one of claim 2, wherein the disinfecting agent is mixed into the process gas downstream of a discharge zone.
